(19) United States
(12) Patent Application Publication
(10) Pub. No.: US 2010/0240595 A1
(43) Pub. Date: Sep. 23, 2010

(54) Improved Antitumoral Treatments

(75) Inventors: Giuseppe Longo Sorbello, New Brunswick, NJ (US); Pravin Jaiprakash Mishra, New Brunswick, NJ (US); Prasun Jaiprakash Mishra, New Brunswick, NJ (US); Joseph Rocco Bertino, New Brunswick, NJ (US); Debabrata Banerjee, New Brunswick, NJ (US); José Maria Jimeno Donaque, Madrid (ES)

Correspondence Address: KING & SPALDING, 1185 AVENUE OF THE AMERICAS, NEW YORK, NY, 10036-4003, US

(73) Assignee: PHARMA MAR ,S.A., Madrid (ES)

(21) Appl. No.: 12/682,910

(22) PCT Filed: Oct. 20, 2008
(86) PCT No.: PCT/EP2008/064117

§ 371 Date: Apr. 14, 2010

Related U.S. Application Data
(60) Provisional application No. 60/981,463, filed on Oct. 19, 2007.

(51) Int. Cl. A61K 38/15; A61P 35/00

(52) U.S. Cl. 514/19.9

(57) ABSTRACT

The present invention relates to combinations of Aplidine with Gemcitabine, and the use of these combinations in the treatment of cancer.

### FIELD OF THE INVENTION

The present invention relates to the combination of Aplidine with other antitumoral agents, in particular with Gemcitabine, and the use of these combinations in the treatment of cancer.

### BACKGROUND OF THE INVENTION

Cancer develops when cells in a part of the body begin to grow out of control. Although there are many kinds of cancer, they all arise from out-of-control growth of abnormal cells. Cancer cells can invade nearby tissues and can spread through the bloodstream and lymphatic system to other parts of the body. There are several main types of cancer. Carcinoma is a malignant neoplasm, which is an uncontrolled and progressive abnormal growth, arising from epithelial cells. Epithelial cells cover internal and external surfaces of the body, including organs, lining of vessels and other small cavities. Sarcoma is cancer arising from cells in bone, cartilage, fat, muscle, blood vessels, or other connective or supportive tissue. Leukemia is cancer that arises in blood-forming tissue such as the bone marrow, and causes large numbers of abnormal blood cells to be produced and enter the bloodstream. Lymphoma and multiple myeloma are cancers that arise from cells of the immune system.

In addition, cancer is invasive and tends to infiltrate the surrounding tissues and give rise to metastases. It can spread directly into surrounding tissues and also may be spread through the lymphatic and circulatory systems to other parts of the body.

Many treatments are available for cancer, including surgery and radiation for localised disease, and chemotherapy. However, the efficacy of available treatments for many cancer types is limited, and new, improved forms of treatment showing clinical benefits are needed. This is especially true for those patients presenting with advanced and/or metastatic disease and for patients relapsing with progressive disease after having been previously treated with established therapies which become ineffective or intolerable due to acquisition of resistance or to limitations in administration of the therapies due to associated toxicities.

Since the 1950s, significant advances have been made in the chemotherapeutic management of cancer. Unfortunately, more than 50% of all cancer patients either do not respond to initial therapy or experience relapse after an initial response to treatment and ultimately die from progressive metastatic disease. Thus, the ongoing commitment to the design and discovery of new anticancer agents is critically important.

Chemotherapy, in its classic form, has been focused primarily on killing rapidly proliferating cancer cells by targeting general cellular metabolic processes, including DNA, RNA, and protein biosynthesis. Chemotherapy drugs are divided into several groups based on how they affect specific chemical substances within cancer cells, which cellular activities or processes the drug interferes with, and which specific phases of the cell cycle the drug affects. The most commonly used types of chemotherapy drugs include: DNA-alkylating drugs (such as cyclophosphamide, ifosfamide, cisplatin, carboplatin, dacarbazine), antimetabolites (5-fluorouracil, capecitabine, 6-mercaptopurine, methotrexate, gemcitabine, cytarabine, fludarabine), mitotic inhibitors (such as paclitaxel, docetaxel, vinblastine, vincristine), anthracyclines (such as daunorubicin, doxorubicin, epirubicin, idarubicin, mitoxantrone), topoisomerase I and II inhibitors (such as topotecan, irinotecan, etoposide, teniposide), and hormone therapy (such as tamoxifen, flutamide).

The ideal antitumor drug would kill cancer cells selectively, with a wide index relative to its toxicity towards non-cancer cells and it would also retain its efficacy against cancer cells, even after prolonged exposure to the drug. Unfortunately, none of the current chemotherapies with these agents possess an ideal profile. Most possess very narrow therapeutic indexes and, in addition, cancerous cells exposed to slightly sublethal concentrations of a chemotherapeutic agent may develop resistance to such an agent, and quite often cross-resistance to several other antitumor agents.

Aplidine (Dehydrodidemnin B) is a cyclic depsipeptide that was isolated from a Mediterranean marine tunicate, Aplidium albicans, and it is the subject of WO 91/04985. It is related to compounds known as didemnins, and has the following structure:

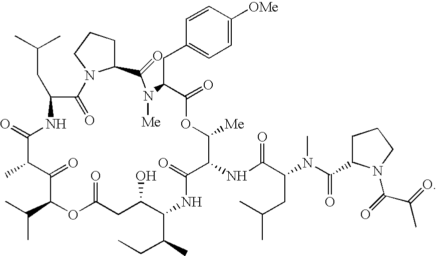

More information on Aplidine, its uses, formulations and synthesis can be found in patent applications WO 91/04985, WO 99/42125, WO 01/35974, WO 01/76616, WO 2004/084812, WO 02/30441, WO 02/02596, WO 03/33013, WO 2004/080477, WO 2004/080421, and WO 2007/101235. We incorporate by specific reference the content of each of these PCT texts.

In both animal preclinical studies and human clinical Phase I studies Aplidine has been shown to have cytotoxic potential against a broad spectrum of tumor types including leukemia and lymphoma. See for example:
Faircloth, G. et al.: “Dehydrodidemnin B (DDB) a new marine derived anticancer agent with activity against experimental tumour models”, 9th NCI-EORTC Symp. New Drugs Cancer Ther. (March 12-15, Amsterdam) 1996, Abst 111;
Faircloth, G. et al.: “Preclinical characterization of aplidine, a new marine anticancer depsipeptide”, Proc. Amer. Assoc. Cancer Res. 1997, 38: Abst 692;
Depenbrock H, Peter R, Faircloth G T, Manzanares I, Jimeno J, Hanauske A R.: “In vitro activity of Aplidine, a new marine-derived anti-cancer compound, on freshly explanted clonogenic human tumour cells and haematopoietic precursor cells” Br. J. Cancer, 1998; 78: 739-744;
Faircloth G, Grant W, Nam S, Jimeno J, Manzanares I, Rinehart K.: “Schedule-dependency of Aplidine, a marine depsipeptide with antitumor activity”, Proc. Am. Assoc. Cancer Res. 1999; 40: 394;
Broggini M, Marchini S, D'Incalci M, Taraboletti G, Giavazzi R, Faircloth G, Jimeno J.: “Aplidine blocks VEGF secretion and VEGF/VEGF-R1 autocrine loop in a human leukemic cell line”, Clin. Cancer Res. 2000; 6 (suppl): 4509;
Erba E, Bassano L, Di Liberti G, Muradore I, Chiorino G, Ubezio P, Vignati S, Codegoni A, Desiderio M A, Faircloth G, Jimeno J and D'Incalci M.: “Cell cycle phase perturbations and apoptosis in tumour cells induced by aplidine”, Br. J. Cancer 2002; 86: 1510-1517;
Paz-Ares L, Anthony A, Pronk L, Twelves C, Alonso S, Cortes-Funes H, Celli N, Gomez C, Lopez-Lazaro L, Guzman C, Jimeno J, Kaye S.: “Phase I clinical and pharmacokinetic study of aplidine, a new marine didemnin, administered as 24-hour infusion weekly” Clin. Cancer Res. 2000; 6 (suppl): 4509;
Raymond E, Ady-Vago N, Baudin E, Ribrag V, Faivre S, Lecot F, Wright T, Lopez Lazaro L, Guzman C, Jimeno J, Ducreux M, Le Chevalier T, Armand J P.: “A phase I and pharmacokinetic study of aplidine given as a 24-hour continuous infusion every other week in patients with solid tumor and lymphoma”, Clin. Cancer Res. 2000; 6 (suppl): 4510;
Maroun J, Belanger K, Seymour L, Soulieres D, Charpentier D, Goel R, Stewart D, Tomiak E, Jimeno J, Matthews S.: “Phase I study of aplidine in a 5 day bolus q 3 weeks in patients with solid tumors and lymphomas”, Clin. Cancer Res. 2000; 6 (suppl): 4509;
Izquierdo M A, Bowman A, Martinez M, Cicchella B, Jimeno J, Guzman C, Germa J, Smyth J.: “Phase I trial of Aplidine given as a 1 hour intravenous weekly infusion in patients with advanced solid tumors and lymphoma”, Clin. Cancer Res. 2000; 6 (suppl): 4509.

Mechanistic studies indicate that Aplidine can block VEGF secretion in ALL-MOLT4 cells and in vitro cytotoxic activity at low concentrations (5 nM) has been observed in AML and ALL samples from pediatric patients with de novo or relapsed ALL and AML. Aplidine appears to induce both a G1 and a G2 arrest in drug treated leukemia cells in vitro. Apart from down regulation of the VEGF receptor, little else is known about the mode(s) of action of Aplidine.

In phase I clinical studies with Aplidine, L-carnitine was given as a 24 hour pretreatment or co-administered to prevent myelotoxicity, see for example WO 02/30441. Co-administration of L-carnitine was proven to be able to improve the recovery of the drug induced muscular toxicity and has allowed for dose escalation of Aplidine.

Previously, in vitro and in vivo assays conducted with Aplidine in combination with other anticancer agents showed that the assayed drug combinations were useful in combination therapy for the treatment of leukemia and lymphoma. In WO 2004/080421, Aplidine was specifically evaluated in combination with methotrexate, cytosine arabinoside, mitoxantrone, vinblastine, methylprednisolone and doxorubicin for the treatment of leukemia and lymphoma. On the other hand, in WO 2007/101235, Aplidine was specifically evaluated in combination with paclitaxel (Taxol®), doxorubicin, cisplatin, arsenic trioxide, 5-fluorouracil (5-FU), cytosine arabinoside (AraC), carboplatin, 7-ethyl-10-hydroxycamptothecin (SN38), etoposide (VP16), melphalan, dexamethasone, cyclophosphamide, bortezomib, erlotinib, trastuzumab, lenalidomide (Revlimid®), interleukin-2 (IL-2), interferon-α 2 (INF-α), dacarbazine (DTIC), bevacizumab (Avastin®), idarubicin, thalidomide, and rituximab for the treatment of lung cancer, breast cancer, colon cancer, prostate cancer, renal cancer, melanoma, multiple myeloma, leukemia and lymphoma.

Since cancer is a leading cause of death in animals and humans, several efforts have been and are still being undertaken in order to obtain an antitumor therapy active and safe to be administered to patients suffering from a cancer. The problem to be solved by the present invention is to provide antitumor therapies that are useful in the treatment of cancer.

### SUMMARY OF THE INVENTION

We have established that Aplidine potentiates other anticancer agents, in particular Gemcitabine, and therefore they can be successfully used in combination therapy for the treatment of cancer. Thus, this invention is directed to pharmaceutical compositions, kits, methods for the treatment of cancer using these combination therapies and uses of Aplidine in the manufacture of a medicament for combination therapy.

In accordance with one aspect of this invention, we provide effective combination therapies for the treatment of cancer based on Aplidine and using Gemcitabine.

In another embodiment, the invention encompasses a method of treating cancer comprising administering to a patient in need of such treatment a therapeutically effective amount of Aplidine, or a pharmaceutically acceptable salt thereof, and a therapeutically effective amount of Gemcitabine, or a pharmaceutically acceptable salt thereof, administered prior, during, or after administering Aplidine. The two drugs may form part of the same composition, or be provided as a separate composition for administration at the same time or at a different time.

In another aspect, the invention encompasses a method of increasing the therapeutic efficacy of Gemcitabine in the treatment of cancer, which comprises administering to a patient in need thereof a therapeutically effective amount of Aplidine, or a pharmaceutically acceptable salt thereof. Aplidine is administered prior, during, or after administering Gemcitabine.

In another embodiment, the invention encompasses the use of Aplidine, or a pharmaceutically acceptable salt thereof, for the manufacture of a medicament for the treatment of cancer, in combination therapy with Gemcitabine.

In a related embodiment, the invention encompasses the use of Gemcitabine, or a pharmaceutically acceptable salt thereof, for the manufacture of a medicament for the treatment of cancer, in combination therapy with Aplidine.

In a further aspect, the invention encompasses a pharmaceutical composition comprising Aplidine, or a pharmaceutically acceptable salt thereof, and/or Gemcitabine, or a pharmaceutically acceptable salt thereof, to be used in combination therapy for the treatment of cancer.

The invention also encompasses a kit for use in the treatment of cancer which comprises a dosage form of Aplidine or a pharmaceutically acceptable salt thereof, and/or a dosage form of Gemcitabine, or a pharmaceutically acceptable salt thereof, and instructions for the use of both drugs in combination.

In one preferred aspect, the present invention is concerned with synergistic combinations of Aplidine or a pharmaceutically acceptable salt thereof, with Gemcitabine, or a pharmaceutically acceptable salt thereof.

### BRIEF DESCRIPTION OF THE FIGURES

FIG. 1A. Dose-effect curve of PANC-1 cells treated with Aplidine (Aplidin)

FIG. 1B. Dose-effect curve of MIA PaCa-2 cells treated with Aplidine (Aplidin)

FIG. 2A. Dose-effect curve of PANC-1 cells treated with Gemcitabine

FIG. 2B. Dose-effect curve of MIA PaCa-2 cells treated with Gemcitabine

FIG. 3. Chou-Talalay analysis of the combination of Aplidine and Gemcitabine in PANC-1 cells

FIG. 4. Chou-Talalay analysis of the combination of Aplidine and Gemcitabine in MIA PaCa-2 cells

FIG. 5. Kinetics of net tumor volume after initiation of treatment with Aplidine (Aplidin) or Gemcitabine (Gem) as single agents or in combination in a pancreatic cancer xenograft model

FIG. 6. Effect of treatment of animals with Aplidine (Aplidin, Apl) and Gemcitabine (Gem) on weight of individual animals

### DETAILED DESCRIPTION OF THE INVENTION

In order to study the possible potentiation of Gemcitabine with Aplidine, we initiated a systematic study firstly to determine the antitumor effect of Aplidine and Gemcitabine when given alone against certain tumor cells, and secondly to determine the existence of any possible synergism between the effect of Aplidine and the effect of Gemcitabine when administered in combination in both in vitro and in vivo studies. As a general conclusion we found that the antitumor activity of Gemcitabine is greatly enhanced in combination with Aplidine. Thus, the present invention is directed to providing an efficacious treatment of cancer based on the combination of Aplidine analogue with Gemcitabine.

By “cancer” it is meant to include tumors, neoplasias, and any other malignant tissue or cells.

In another aspect, the invention relates to synergistic combinations employing Aplidine, or a pharmaceutically acceptable salt thereof, and Gemcitabine, or a pharmaceutically acceptable salt thereof. An indication of synergy can easily be obtained by testing combinations and analyzing the results, for example by the Chou-Talalay method. Reference is made to Example 2 to illustrate this point.

The term “combination” as used throughout the specification, is meant to encompass the administration of the therapeutic agents in the same or separate pharmaceutical formulations, and at the same time or at different times. If the therapeutic agents are administered at different times they should be administered sufficiently close in time to provide for the synergistic response to occur.

In another aspect, the invention is directed to the use of Aplidine, or a pharmaceutically acceptable salt thereof, for the manufacture of a medicament for an effective treatment of cancer by combination therapy employing Aplidine, or a pharmaceutically acceptable salt thereof, with Gemcitabine, or a pharmaceutically acceptable salt thereof.

In a related aspect, the invention is directed to the use of Gemcitabine, or a pharmaceutically acceptable salt thereof, for the manufacture of a medicament for an effective treatment of cancer by combination therapy employing Gemcitabine, or a pharmaceutically acceptable salt thereof, with Aplidine, or a pharmaceutically acceptable salt thereof.

In a further aspect, the present invention is directed to a method of treating cancer comprising administering to a patient in need of such treatment a therapeutically effective amount of Aplidine, or a pharmaceutically acceptable salt thereof, in combination with a therapeutically effective amount of Gemcitabine, or a pharmaceutically acceptable salt thereof.

The invention also provides a method of treating cancer comprising administering a therapeutically effective amount of Gemcitabine, or a pharmaceutically acceptable salt thereof, in combination with a therapeutically effective amount of Aplidine, or a pharmaceutically acceptable salt thereof.

As mentioned above, Aplidine is a cyclic depsipeptide with the following structure:

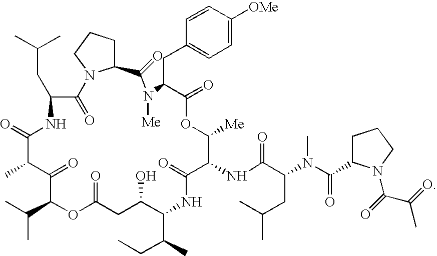

The term “Aplidine” is intended here to cover any pharmaceutically acceptable salt, ester, solvate, hydrate, prodrug, or any other compound which, upon administration to the patient is capable of providing (directly or indirectly) the compounds as described herein. However, it will be appreciated that non-pharmaceutically acceptable salts also fall within the scope of the invention since these may be useful in the preparation of pharmaceutically acceptable salts. The preparation of salts, esters, solvates, hydrates, and prodrugs can be carried out by methods known in the art.

Pharmaceutically acceptable salts of Aplidine are synthesized from the parent compound, which contains a basic or acidic moiety, by conventional chemical methods. Generally, such salts are, for example, prepared by reacting the free acid or base forms of these compounds with a stoichiometric amount of the appropriate base or acid in water or in an organic solvent or in a mixture of the two. Generally, nonaqueous media like ether, ethyl acetate, ethanol, isopropanol or acetonitrile are preferred. Examples of the acid addition salts include mineral acid addition salts such as, for example, hydrochloride, hydrobromide, hydroiodide, sulphate, nitrate, phosphate, and organic acid addition salts such as, for example, acetate, trifluoroacetate, maleate, fumarate, citrate, oxalate, succinate, tartrate, malate, mandelate, methanesulphonate and p-toluenesulphonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine and basic aminoacids salts.

In addition, Aplidine may be in crystalline form either as free compound or as solvates (e.g. hydrates) and it is intended that both forms are within the scope of the present invention. Methods of solvation are generally known within the art.

Any compound that is a prodrug of Aplidine is within the scope and spirit of the invention. The term “prodrug” is used in its broadest sense and encompasses those derivatives that are converted in vivo to Aplidine. The prodrug can hydrolyze, oxidize, or otherwise react under biological conditions to provide Aplidine. Such derivatives would readily occur to those skilled in the art, and include, for example, compounds where a free hydroxy group is converted into an ester derivative.

Any compound referred to herein is intended to represent such specific compound as well as certain variations or forms. In particular, compounds referred to herein may have asymmetric centres and therefore exist in different enantiomeric forms. All optical isomers and stereoisomers of the compounds referred to herein, and mixtures thereof, are considered within the scope of the present invention. Thus any given compound referred to herein is intended to represent any one of a racemate, one or more enantiomeric forms, one or more diastereomeric forms, one or more atropisomeric forms, and mixtures thereof. Particularly, the compounds of the present invention may include enantiomers depending on their asymmetry or diastereoisomers. Stereoisomerism about the double bond is also possible, therefore in some cases the molecule could exist as (E)-isomer or (Z)-isomer. If the molecule contains several double bonds, each double bond will have its own stereoisomerism, that could be the same or different than the stereoisomerism of the other double bonds of the molecule. The single isomers and mixtures of isomers fall within the scope of the present invention.

Furthermore, compounds referred to herein may exist as geometric isomers (i.e., cis and trans isomers), as tautomers, or as atropisomers. Specifically, the term tautomer refers to one of two or more structural isomers of a compound, that exist in equilibrium and are readily converted from one isomeric form to another. Common tautomeric pairs are amine-imine, amide-imide, keto-enol, lactam-lactim, etc. Additionally, any compound referred to herein is intended to represent hydrates, solvates, and polymorphs, and mixtures thereof when such forms exist in the medium. In addition, compounds referred to herein may exist in isotopically-labelled forms. All geometric isomers, tautomers, atropisomers, hydrates, solvates, polymorphs, and isotopically labelled forms of the compounds referred to herein, and mixtures thereof, are considered within the scope of the present invention.

Aplidine for use in accordance of the present invention may be prepared following a synthetic process such as those disclosed in WO 02/02596, WO 01/76616, and WO 2004/084812, which are incorporated herein by reference.

Pharmaceutical compositions of Aplidine that can be used include solutions, suspensions, emulsions, lyophilised compositions, etc., with suitable excipients for intravenous administration. Preferably, Aplidine may be supplied and stored as a sterile lyophilized product, comprising Aplidine and excipients in a formulation adequate for therapeutic use. In particular a formulation comprising mannitol is preferred. Further guidance on Aplidine formulations is given in WO 99/42125 which is incorporated herein by reference in its entirety.

Administration of Aplidine, or pharmaceutical compositions thereof, is preferably by intravenous infusion. We prefer that infusion times of up to 72 hours are used, more preferably 1 to 24 hours, with about 1, about 3 or about 24 hours most preferred. Short infusion times which allow treatment to be carried out without an overnight stay in hospital are especially desirable. However, infusion may be around 24 hours or even longer if required. Infusion may be carried out at suitable intervals with varying patterns, illustratively once a week, twice a week, or more frequently per week, repeated each week optionally with gaps of typically one or several weeks.

Gemcitabine is a nucleoside analogue with the following structural formula:

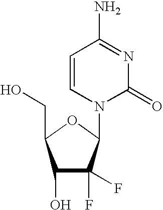

This drug is being marketed in the form of its hydrochloride salt with the trade name Gemzar®. This drug is currently indicated for the treatment of certain types of cancer, specifically for ovarian cancer, breast cancer, non-small cell lung cancer (NSCLC) and pancreatic cancer. As single agent, Gemcitabine is recommended to be administered by intravenous infusion at a dose of 1000 mg/m² over 30 minutes once weekly for up to 7 weeks, followed by a week of rest from treatment. Subsequent cycles should consist of infusions once weekly for 3 consecutive weeks out of every 4 weeks. Information about this drug is available on the website www.gemzar.com and the extensive literature on Gemcitabine.

Gemcitabine exhibits cell phase specificity, primarily killing cells undergoing DNA synthesis (S-phase) and also blocking the progression of cells through the G1/S-phase boundary. Gemcitabine is metabolized intracellularly by nucleoside kinases to the active diphosphate (dFdCDP) and triphosphate (dFdCTP) nucleosides. The cytotoxic effect of Gemcitabine is attributed to a combination of two actions of the diphosphate and the triphosphate nucleosides, which leads to inhibition of DNA synthesis. First, Gemcitabine diphosphate inhibits ribonucleotide reductase, which is responsible for catalyzing the reactions that generate the deoxynucleoside triphosphates for DNA synthesis. Inhibition of this enzyme by the diphosphate nucleoside causes a reduction in the concentrations of deoxynucleotides, including dCTP. Second, Gemcitabine triphosphate competes with dCTP for incorporation into DNA. The reduction in the intracellular concentration of dCTP (by the action of the diphosphate) enhances the incorporation of Gemcitabine triphosphate into DNA (self-potentiation). After the Gemcitabine nucleotide is incorporated into DNA, only one additional nucleotide is added to the growing DNA strands. After this addition, there is inhibition of further DNA synthesis. DNA polymerase epsilon is unable to remove the Gemcitabine nucleotide and repair the growing DNA strands (masked chain termination). In CEM T lymphoblastoid cells, Gemcitabine induces internucleosomal DNA fragmentation, one of the characteristics of programmed cell death.

Aplidine, or a pharmaceutically acceptable salt thereof, and Gemcitabine, or a pharmaceutically acceptable salt thereof, may be provided as separate medicaments for administration at the same time or at different times. Preferably, Aplidine, or a pharmaceutically acceptable salt thereof, and Gemcitabine, or a pharmaceutically acceptable salt thereof, are provided as separate medicaments for administration at different times. When administered separately and at different times, either Aplidine, or a pharmaceutically acceptable salt thereof, or Gemcitabine, or a pharmaceutically acceptable salt thereof, may be administered first. In addition, both drugs can be administered in the same day or at different days, and they can be administered using the same schedule or at different schedules during the treatment cycle. Thus, the pharmaceutical compositions of the present invention may comprise all the components (drugs) in a single pharmaceutically acceptable formulation. Alternatively, the components may be formulated separately and administered in combination with one another. Various pharmaceutically acceptable formulations well known to those of skill in the art can be used in the present invention.

Preferably, a combination of Aplidine, or a pharmaceutically acceptable salt thereof, and Gemcitabine, or a pharmaceutically acceptable salt thereof, can be used in any suitable formulation for combined or separate intravenous administration. The intravenous formulations of the combination may include solutions, suspensions, emulsions, lyphilised compositions, and the like. However, selection of an appropriate formulation for use in the present invention can be performed routinely by those skilled in the art based upon the mode of administration and the solubility characteristics of the components of the composition.

The correct dosage of the compounds of the combination will vary according to the particular formulation, the mode of application, and the particular site, host and tumour being treated. Other factors like age, body weight, sex, diet, time of administration, rate of excretion, condition of the host, drug combinations, reaction sensitivities and severity of the disease shall be taken into account. Administration can be carried out continuously or periodically within the maximum tolerated dose. Further guidance for the administration of Aplidine is given in WO 01/35974 which is incorporated herein by reference in its entirety.

In another aspect, the present invention is directed to a kit for administering Aplidine in combination with Gemcitabine in the treatment of cancer, comprising a supply of Aplidine, or a pharmaceutically acceptable salt thereof, in dosage units for at least one cycle, and printed instructions for the use of both drugs in combination.

In a related aspect, the present invention is directed to a kit for administering Gemcitabine in combination with Aplidine in the treatment of cancer, comprising a supply of Gemcitabine, or a pharmaceutically acceptable salt thereof, in dosage units for at least one cycle, and printed instructions for the use of both drugs in combination.

In a related aspect, the present invention is directed to a kit for administering Aplidine in combination with Gemcitabine in the treatment of cancer, comprising a supply of Aplidine, or a pharmaceutically acceptable salt thereof, in dosage units for at least one cycle, a supply of Gemcitabine, or a pharmaceutically acceptable salt thereof, in dosage units for at least one cycle, and printed instructions for the use of both drugs in combination.

In another aspect, the present invention also provides a pharmaceutical composition comprising Aplidine or a pharmaceutically acceptable salt thereof, and a pharmaceutically acceptable carrier, for use in combination with Gemcitabine in the treatment of cancer.

In a further aspect, the present invention also provides a pharmaceutical composition comprising Gemcitabine, or a pharmaceutically acceptable salt thereof, and a pharmaceutically acceptable carrier, for use in combination with Aplidine in the treatment of cancer.

In addition, the present invention also provides a pharmaceutical composition comprising Aplidine, or a pharmaceutically acceptable salt thereof, Gemcitabine, or a pharmaceutically acceptable salt thereof, and a pharmaceutically acceptable carrier, for use in the treatment of cancer.

In another aspect, the invention further provides for the use of Aplidine, or a pharmaceutically acceptable salt thereof, in the preparation of a composition for use in combination with Gemcitabine in the treatment of cancer.

In a related aspect, the invention further provides for the use of Gemcitabine, or a pharmaceutically acceptable salt thereof, in the preparation of a composition for use in combination with Aplidine in the treatment of cancer.

And in a further aspect, the invention also provides for the use of Aplidine, or a pharmaceutically acceptable salt thereof, and Gemcitabine, or a pharmaceutically acceptable salt thereof, in the preparation of a composition for use in the treatment of cancer.

In another aspect, the invention further provides for the use of Aplidine or a pharmaceutically acceptable salt thereof, for the manufacture of a medicament for the treatment of cancer, in combination therapy with Gemcitabine.

In a related aspect, the invention further provides for the use of Gemcitabine, or a pharmaceutically acceptable salt thereof, for the manufacture of a medicament for the treatment of cancer, in combination therapy with Aplidine.

In a related aspect, the invention further provides for the use of Aplidine, or a pharmaceutically acceptable salt thereof, in combination with Gemcitabine, or a pharmaceutically acceptable salt thereof, for the manufacture of a medicament for the treatment of cancer.

In another aspect, the invention further provides for the use of Aplidine, or a pharmaceutically acceptable salt thereof, for the treatment of cancer, in combination therapy with Gemcitabine.

In a related aspect, the invention further provides for the use of Gemcitabine, or a pharmaceutically acceptable salt thereof, for the treatment of cancer, in combination therapy with Aplidine.

In another aspect, the invention further provides for the use of Aplidine, or a pharmaceutically acceptable salt thereof, in combination with Gemcitabine, or a pharmaceutically acceptable salt thereof, for the treatment of cancer.

In another aspect, the invention further provides for the use of Aplidine, or a pharmaceutically acceptable salt thereof, as a medicament, in combination therapy with Gemcitabine.

In a related aspect, the invention further provides for the use of Gemcitabine, or a pharmaceutically acceptable salt thereof, as a medicament, in combination therapy with Aplidine.

In another aspect, the invention further provides for the use of Aplidine, or a pharmaceutically acceptable salt thereof, in combination with Gemcitabine, or a pharmaceutically acceptable salt thereof, as a medicament.

In another aspect, the invention further provides for the use of Aplidine, or a pharmaceutically acceptable salt thereof, as a medicament for the treatment of cancer, in combination therapy with Gemcitabine, or pharmaceutically acceptable salt thereof.

In a related aspect, the invention further provides for the use of Gemcitabine, or a pharmaceutically acceptable salt thereof, as a medicament for the treatment of cancer, in combination therapy with Aplidine, or pharmaceutically acceptable salt thereof.

In another aspect, the invention further provides for the use of Aplidine, or a pharmaceutically acceptable salt thereof, in combination with Gemcitabine, or a pharmaceutically acceptable salt thereof, as a medicament for the treatment of cancer.

In another aspect, the invention provides Aplidine, or a pharmaceutically acceptable salt thereof, for the treatment of cancer comprising administering a therapeutically effective amount of Aplidine, or a pharmaceutically acceptable salt thereof, in combination with a therapeutically effective amount of Gemcitabine, or a pharmaceutically acceptable salt thereof.

In a related aspect, the invention further provides Gemcitabine, or a pharmaceutically acceptable salt thereof, for the treatment of cancer comprising administering a therapeutically effective amount of Gemcitabine, or a pharmaceutically acceptable salt thereof, in combination with a therapeutically effective amount of Aplidine, or a pharmaceutically acceptable salt thereof.

In another aspect, the invention provides for the treatment of cancer comprising the administration of therapeutically effective amounts of Aplidine, or pharmaceutically acceptable salt thereof, in combination with the administration of therapeutically effective amounts of Gemcitabine, or a pharmaceutically acceptable salt thereof, wherein the combination may be administered together or separately.

Depending on the type of tumor and the development stage of the disease, the treatments of the invention are useful in promoting tumor regression, in stopping tumor growth and/or in preventing metastasis. In particular, the method of the invention is suited for human patients, especially those who are relapsing or refractory to previous chemotherapy. First line therapy is also envisaged.

Preferably, the combination of Aplidine, or a pharmaceutically acceptable salt thereof, with Gemcitabine, or a pharmaceutically acceptable salt thereof, is used for the treatment of pancreatic cancer, bladder cancer, non small cell lung cancer, renal cancer, and colorectal cancer. Specially preferred is the use of the combination for the treatment of pancreatic cancer and bladder cancer.

In one embodiment, cancer cells are contacted, or otherwised treated, with a combination of Aplidine, or a pharmaceutically acceptable salt thereof, and Gemcitabine, or a pharmaceutically acceptable salt thereof. The cancer cells are preferably human and may include carcinoma cells, sarcoma cells, leukemia cells, lymphoma cells and myeloma cells. More preferably, the cancer cells may include pancreatic cancer cells, bladder cancer cells, non small cell lung cancer cells, colorectal cancer cells, and renal cancer cells. In particular, the cancer cells may include human pancreatic carcinoma cells. In addition, the combination may provide a synergistic inhibitory effect against cancer cells, particularly against human pancreatic carcinoma cells. For example, the cancer cells may be in culture and the combination may be administered in vitro. The combination may be delivered together or separately. A lower level of proliferation or survival of the contacted cancer cells in culture compared to the non-contacted cancer cells in culture suggests that the combination of Aplidine, or a pharmaceutically acceptable salt thereof, and Gemcitabine, or a pharmaceutically acceptable salt thereof, may be effective for treating a patient with that particular type of cancer.

In another embodiment, the combination of Aplidine, or a pharmaceutically acceptable salt thereof, and Gemcitabine, or a pharmaceutically acceptable salt thereof, may inhibit tumor growth or reduce the size of a tumor in vivo. In particular, the combination may inhibit in vivo growth of carcinoma cells, sarcoma cells, leukemia cells, lymphoma cells and myeloma cells. Preferably, the combination may inhibit in vivo growth of pancreatic cancer cells, bladder cancer cells, non small cell lung cancer cells, colorectal cancer cells, and renal cancer cells. Specifically, the combination may inhibit in vivo growth of human pancreatic carcinoma cells. Similarly, the combination may reduce the size of carcinoma, sarcoma, leukemia, lymphoma and myeloma tumors in vivo. Preferably, the combination may reduce the size of pancreatic cancer, bladder cancer, non small cell lung cancer, colorectal cancer, and renal cancer tumors in vivo. Specifically, the combination may reduce the size of human pancreatic carcinoma tumors in vivo.

For example, the combination may inhibit tumor growth or reduce the size of human cancer xenografts, particularly human pancreatic carcinoma xenografts, in animal models. A reduced growth or reduced size of human cancer xenografts in animal models administered with the combination suggests that the combination of Aplidine, or a pharmaceutically acceptable salt thereof, and Gemcitabine, or a pharmaceutically acceptable salt thereof, may be effective for treating a patient with that particular type of cancer. In addition, a low level of toxicity in animal models suggests that the combination may provide selective cytotoxicity against cancer cells, particularly against human pancreatic carcinoma cells.

In another aspect, the invention provides for a method for inhibiting the growth of cancer cells comprising contacting said cancer cells with an effective amount of Aplidine, or a pharmaceutically acceptable salt thereof, in combination with Gemicitabine.

In a related aspect, the invention provides for a method for inhibiting the growth of cancer cells comprising contacting said cancer cells with an effective amount of Gemicitabine, or a pharmaceutically acceptable salt thereof, in combination with Aplidine.

In a related aspect, the invention provides for a method for inhibiting the growth of cancer cells comprising contacting said cancer cell with an effective combination of Aplidine, or a pharmaceutically acceptable salt thereof, and Gemcitabine, or a pharmaceutically acceptable salt thereof, together or separately.

In another aspect, the invention provides for a method for inhibiting the growth of cancer cells comprising contacting said cancer cell with a synergistic combination of Aplidine, or a pharmaceutically acceptable salt thereof, and Gemcitabine, or a pharmaceutically acceptable salt thereof, together or separately, wherein said combination provides improved inhibition against cancer cell growth as compared to (i) Aplidine, or a pharmaceutically acceptable salt thereof, in the absence of Gemcitabine or (ii) Gemcitabine, or pharmaceutically acceptable salt thereof, in the absence of Aplidine.

In another aspect, the invention provides for a pharmaceutical composition comprising an effective amount of Aplidine, or a pharmaceutically acceptable salt thereof, for use in combination with Gemicitabine for inhibiting the growth of cancer cells.

In a related aspect, the invention provides for a pharmaceutical composition comprising an effective amount of Gemicitabine, or a pharmaceutically acceptable salt thereof, for use in combination with Aplidine for inhibiting the growth of cancer cells.

In a related aspect, the invention provides for a pharmaceutical composition comprising an effective combination of Aplidine, or a pharmaceutically acceptable salt thereof, and Gemcitabine, for inhibiting the growth of cancer cells.

In another aspect, the invention provides for a pharmaceutical composition comprising a synergistic combination of Aplidine, or a pharmaceutically acceptable salt thereof, and Gemcitabine, or a pharmaceutically acceptable salt thereof, for inhibiting the growth of cancer cells, wherein said combination provides improved inhibition against cancer cell growth as compared to (i) Aplidine, or a pharmaceutically acceptable salt thereof, in the absence of Gemcitabine or (ii) Gemcitabine, or pharmaceutically acceptable salt thereof, in the absence of Aplidine.

In another aspect, the invention provides for a method for reducing the size of a tumor, comprising administering an effective amount of Aplidine, or a pharmaceutically acceptable salt thereof, in combination with Gemicitabine.

In a related aspect, the invention provides for a method for reducing the size of a tumor, comprising administering an effective amount of Gemicitabine, or a pharmaceutically acceptable salt thereof, in combination with Aplidine.

In a related aspect, the invention provides for a method for reducing the size of a tumor, comprising administering an effective combination of Aplidine, or a pharmaceutically acceptable salt thereof, and Gemcitabine, or a pharmaceutically acceptable salt thereof, together or separately.

In another aspect, the invention provides for a cytotoxic composition comprising an effective amount of Aplidin, or a pharmaceutically acceptable salt thereof, for use in combination with Gemcitabine, wherein the composition is selectively cytotoxic against cancer cells.

In a related aspect, the invention provides for a cytotoxic composition comprising an effective amount of Gemcitabine, or a pharmaceutically acceptable salt thereof, for use in combination with Aplidine, wherein the composition is selectively cytotoxic against cancer cells.

In a related aspect, the invention provides for a cytotoxic composition comprising an effective combination of Aplidin, or a pharmaceutically acceptable salt thereof, and Gemcitabine, or a pharmaceutically acceptable salt thereof, wherein the composition is selectively cytotoxic against cancer cells.

The following example further illustrates the invention. It should not be interpreted as a limitation of the scope of the invention.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

### Examples

### Example 1

### Determination of Aplidine and Gemcitabine in vitro Cytotoxicity in Human Pancreas Carcinoma Cell Lines

PANC-1 (ATCC CRL-1469) and MIA PaCa-2 (ATCC CRL-1420) cell lines obtained from ATCC (Rockville, Md.) were used to check the cytotoxicity of Aplidine and Gemcitabine hydrochloride salt in human pancreas carcinoma cell lines. These cell lines were maintained in Dulbecco's modified Eagle medium with 4 mmol/L glutamine containing 10% Fetal Bovine Serum (FBS), and 1% Penicillin/Streptomycin solution, at 37° C. in humidified atmosphere of 5% CO₂.

Four thousand cells/well were plated in a 96/wells tissue culture plate and exposed to different concentrations of Aplidine or Gemcitabine for 96 hours at 37° C. and 5% CO₂. At the end of the incubation the viability of the cells was tested with the MTS/PMS assay (Riss T L and Moravec R A, Mol. Biol. Cell, 1992, 3, 184a). Cell viability was correlated to the amount of formazan quantified spectrophotometrically at 450 nm (reference wavelength 670 nm) using a microplate reader (SpectraMax® Plus³⁸⁴, Molecular Devices, Sunnyvale, Calif.).

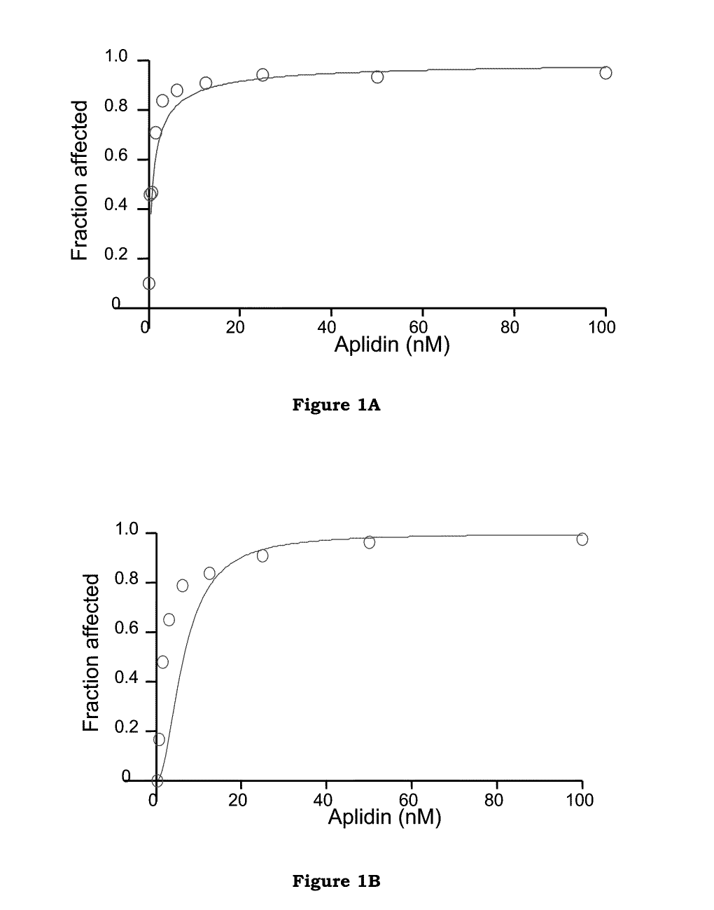

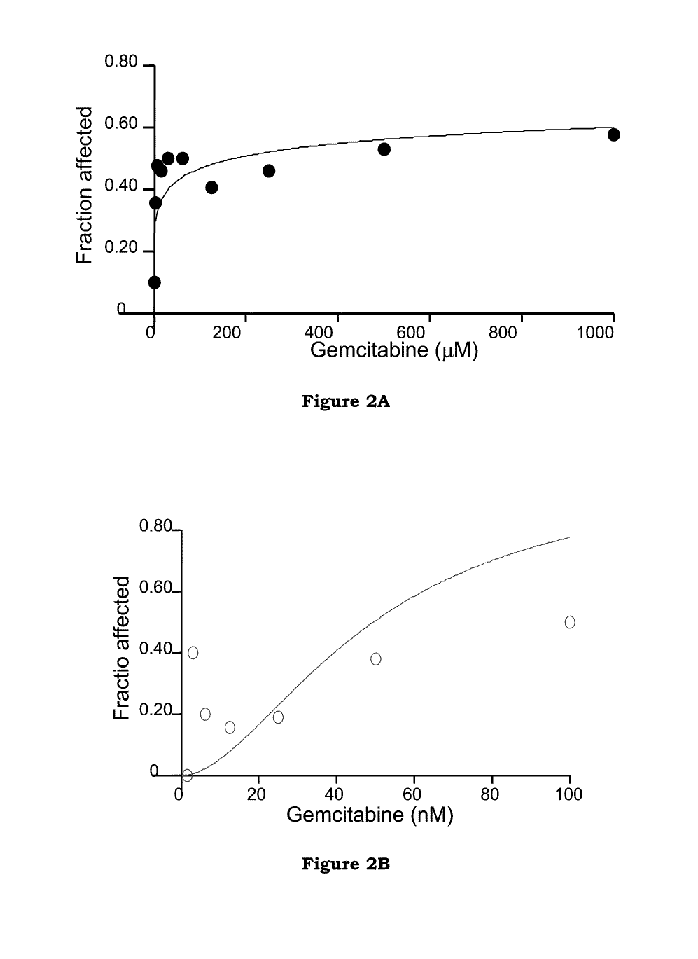

IC₅₀ values of Aplidine and Gemcitabine were 1 nM and 1 μM, respectively, against PANC-1 cell line, and 1 nM and 150 nM, respectively, against MIA PaCa-2 cell line. In addition, FIGS. 1A and 1B disclose the dose-effect curve of PANC-1 and MIA PaCa-2 cell lines, respectively, treated with Aplidine, and FIGS. 2A and 2B disclose the dose-effect curve of PANC-1 and MIA PaCa-2 cell lines, respectively, treated with Gemcitabine. Data shown in this study are means of three experiments±SD.

### Example 2

### Determination of the In Vitro Effect of Aplidine in Combination with Gemcitabine in Human Pancreas Carcinoma Cell Lines

The in vitro effect of the combination of Aplidine with Gemcitabine was tested against two different human pancreas carcinoma cell lines: PANC-1 and MIA PaCa-2.

When the combination was tested against PANC-1 cell line, Aplidine was combined with Gemcitabine hydrochloride salt, at a fixed ratio of Aplidine doses that corresponded to 0.0078, 0.015625, 0.03125, 0.0625, 0.125, 0.25, 0.5, and 1 times the individual IC₅₀ value for Aplidine alone, and at a fixed ratio of Gemcitabine doses that corresponded to 0.78, 1.5, 3.1, 6.2, 12.5, 25, 50 and 100 times the individual IC50 value for Gemcitabine alone.

When the combination was tested against MIA PaCa-2 cell line, Aplidine was combined with Gemcitabine hydrochloride salt, at a fixed ratio of doses that corresponded to 0.031, 0.062, 0.125, 0.25, 0.5, and 1 times the individual IC₅₀ values for each drug alone.

The methodology and assay conditions were identical to those of Example 1 wherein Aplidine and Gemcitabine hydrochloride salt were tested as single agents. Briefly, the drugs used in the combination study were added at ratios that reflected a ratio of their IC₅₀ (50% of concentration required for 100% cell kill), followed by 96 h incubation with the drugs.

The combination index (CI) was calculated based on the Chou-Talalay equation, which takes into account both potency (Dm or IC₅₀) and the shape of the dose-effect curve. CI<1, CI=1, CI>1 indicate synergism, additive effect, and antagonism, respectively (Chou T C and Talalay P. Adv. Enzyme Regul. 1984, 22, 27-55). CalcuSyn software (Biosoft, Ferguson, Mo.) was used for the Chou-Talalay combination index analysis.

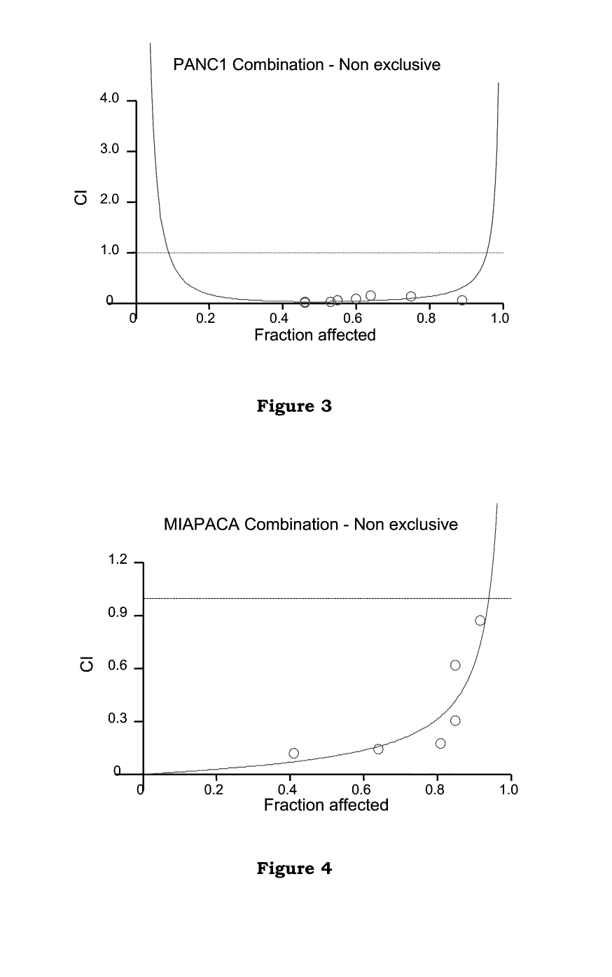

Table 1 provides the Combination Index (CI) that was obtained when combining Aplidine with Gemcitabine at different doses on PANC-1 cells. Synergism was observed in all the doses tested of the combination (FIG. 3).

TABLE 1

| Aplidine (nM) | Gemcitabine (nM) | CI |
|---|---|---|
| 0.0078 | 780 | 0.021 |
| 0.015 | 1500 | 0.041 |
| 0.031 | 3100 | 0.044 |
| 0.062 | 6200 | 0.074 |
| 0.125 | 12500 | 0.103 |
| 0.25 | 25000 | 0.157 |
| 0.5 | 50000 | 0.146 |
| 1 | 100000 | 0.077 |

Table 2 provides the Combination Index (CI) that was obtained when combining Aplidine with Gemcitabine at different doses on MIA PaCa-2 cells. Synergism was also observed in all the doses tested of the combination (FIG. 4).

TABLE 2

| Aplidin (nM) | Gemcitabine (nM) | CI |
|---|---|---|
| 0.031 | 4.65 | 0.41 |
| 0.062 | 9.3 | 0.64 |
| 0.125 | 18.75 | 0.81 |
| 0.25 | 37.5 | 0.85 |
| 0.5 | 75 | 0.85 |
| 1 | 150 | 0.92 |

This data demonstrates a profound synergism between Aplidin and Gemcitabine when combined.

The term “Non exclusive”, which appears in FIGS. 3 and 4, is used in the context of the Chou-Talalay combination index analysis for synergism, antagonism or additivity. Two or more drugs when used in combination on cells are considered to be non-exclusive when they have independent modes of action. Observed synergism values are usually underestimated and antagonism is usually overestimated by this criteria. Thus, synergism is anticipated to be greater than calculated values.

### Example 3

### Determination of the In Vivo Effect of Aplidine in Combination with Gemcitabine in a Pancreatic Cancer Xenograft Model

Pathogen-free NCR Nu/Nu mice, of 5 to 6 weeks old and purchased from Taconic Farms (Germantown, N.Y.), were housed in microisolator cages under specific pathogen-free conditions. The animals were provided autoclaved food and water ad libitum.

Mice were inoculated subcutaneously in the right flank with 0.5×10⁷ PANC-1 cells containing 10% matrigel. After establishment of palpable tumors, animals were randomized into 6 groups:
A first group of 5 animals remained untreated and they just received the vehicle used for the administration of Aplidine, which contained 15% Cremophor EL/15% Ethanol/70% WFI diluted in saline.
A second group of 5 mice were treated with Aplidine alone (0.6 mg/kg/wk on days 1 and 8).
A third group of 5 mice were treated with Gemcitabine HCl alone (250 mg/kg on days 1, 4, 8 and 12).
A fourth group of 8 mice were treated with 250 mg/kg of Gemcitabine HCl on days 1, 4, 8 and 12, and 0.2 mg/kg of Aplidine on days 1 and 8.
A fifth group of 8 mice were treated with 250 mg/kg of Gemcitabine HCl on days 1, 4, 8 and 12, and 0.3 mg/kg of Aplidine on days 1 and 8.
A sixth group of 8 mice were treated with 250 mg/kg of Gemcitabine HCl on days 1, 4, 8 and 12, and 0.4 mg/kg of Aplidine on days 1 and 8.

As mentioned before, the vehicle used for the administration of Aplidine contained 15% Cremophor EL/15% Ethanol/70% WFI diluted in saline, whereas Gemcitabine was prepared in saline in order to be administered.

Both drugs were administered intra-peritoneally. On days when both drugs were administered in Groups 4-6 (on days 1 and 8), Aplidine was injected first to all eight animals in the group, followed 30 minutes later by the administration of Gemcitabine. Tumor size and weight of the animals were recorded every other day, starting from the first day of treatment. Tumor volume was calculated according to the formula:

tumor volume (mm³)=(longer diameter)×[(shorter diameter)²]/2.

Comparison of the median tumor volume in the treatment groups (T) to the median tumor volume in the control group (C) [T/C] on day 20 was used for evaluation of tumor efficacy. % T/C [T/C×100%] for each treatment is reported in Table 3. Interestingly, the lower dose (0.2 mg/kg) of Aplidine in combination with Gemcitabine (at 250 mg/kg) was the most potent, demonstrating a % T/C on day 20 of 7%.

TABLE 3

| Group | | | % T/C on |
|---|---|---|---|
| No. | Agent | Dosage | day 20 |
| 1 | 15% Cremophor | 0 mg/kg | — |
| | EL/15% Ethanol/ | | |
| | 70% WFI diluted | | |
| | in saline | | |
| 2 | Aplidine | 0.6 mg/kg on day 1 and 8 | 23 |
| 3 | Gemcitabine | 250 mg/kg on day 1, 4, 8 & | 45 |
| | | 12 | |
| 4 | Aplidine/ | 0.2 mg/kg on day 1 & 8/ | 7 |
| | Gemcitabine | 250 mg/kg on day 1, 4, 8 & | |
| | | 12 | |
| 5 | Aplidine/ | 0.3 mg/kg on day 1 & 8/ | 17 |
| | Gemcitabine | 250 mg/kg on day 1, 4, 8 & | |
| | | 12 | |
| 6 | Aplidine/ | 0.4 mg/kg on day 1 & 8/ | 16 |
| | Gemcitabine | 250 mg/kg on day 1, 4, 8& | |
| | | 12 | |

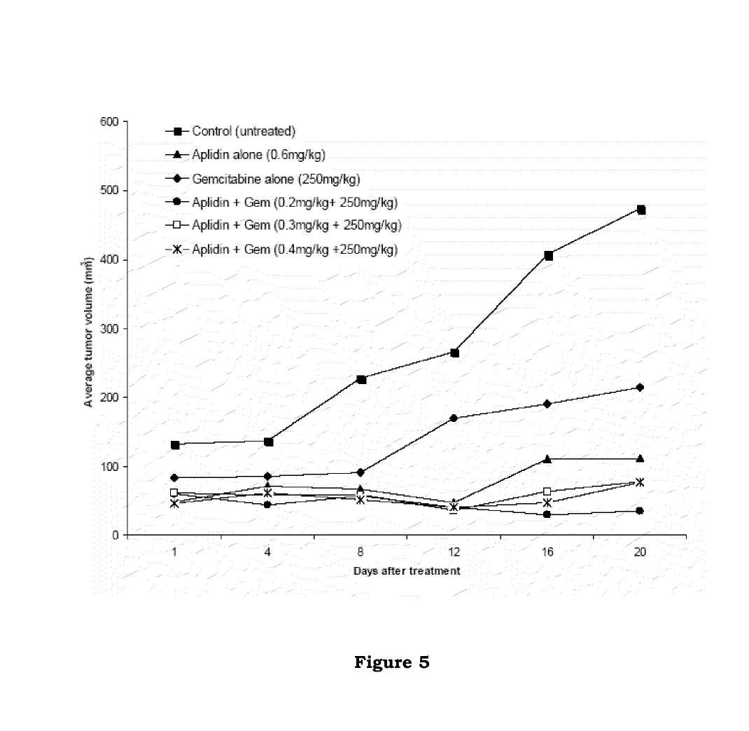

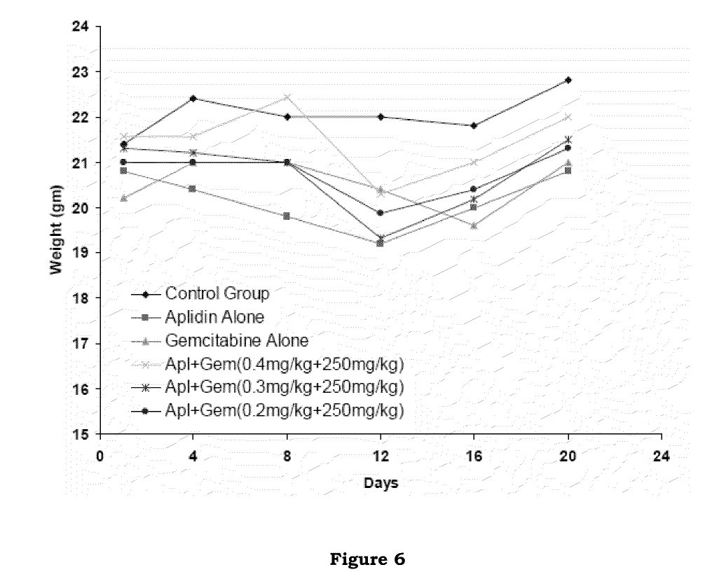

FIG. 5 shows kinetics of net tumor volume after initiation of treatment with Aplidine or Gemcitabine as single agents or in combination at different doses of Aplidine. FIG. 6 shows that animals treated with the combination of Aplidine and Gemcitabine do not exhibit toxicity as reflected by negligible weight loss following treatment. Animals that showed some weight loss recovered quickly.

From these data it can be concluded that Aplidine clearly potentiates the antitumoral effect of Gemcitabine. As can be seen, all three Aplidine doses combined with a fixed dose of Gemcitabine were more effective than either of the drugs alone. In addition, the combination of Aplidine and Gemcitabine did not show any significant toxicity. In addition, because the results show that Gemcitabine and Aplidine do not have overlapping toxicities, the combination of these two drugs may provide a higher therapeutic index.

1-20. (canceled)

21. A method of treating cancer comprising administering to a patient in need of such treatment a therapeutically effective amount of Aplidine, or a pharmaceutically acceptable salt thereof, and a therapeutically effective amount of Gemcitabine, or a pharmaceutically acceptable salt thereof.

22. A method of increasing the therapeutic efficacy of Gemcitabine in the treatment of cancer, which comprises administering to a patient in need thereof, Gemcitabine and a therapeutically effective amount of Aplidine, or a pharmaceutically acceptable salt thereof.

23. The method according to claim 21, wherein the cancer to be treated is selected from pancreatic cancer, bladder cancer, non small cell lung cancer, colorectal cancer, and renal cancer.

24. The method according to claim 22, wherein the cancer to be treated is selected from pancreatic cancer, bladder cancer, non small cell lung cancer, colorectal cancer, and renal cancer.

25. The method according to claim 23, wherein Aplidine, or a pharmaceutically acceptable salt thereof, and Gemcitabine, or a pharmaceutically acceptable salt thereof, form part of the same composition.

26. The method according to claim 23, wherein Aplidine, or a pharmaceutically acceptable salt thereof, and Gemcitabine, or a pharmaceutically acceptable salt thereof, are provided as separate compositions for administration at the same time or at different times.

27. The method according to claim 26, wherein Aplidine, or a pharmaceutically acceptable salt thereof, and Gemcitabine, or a pharmaceutically acceptable salt thereof, are provided as separate compositions for administration at different times.

28. The method according to claim 24, wherein Aplidine, or a pharmaceutically acceptable salt thereof, and Gemcitabine, or a pharmaceutically acceptable salt thereof, form part of the same composition.

29. The method according to claim 24, wherein Aplidine, or a pharmaceutically acceptable salt thereof, and Gemcitabine, or a pharmaceutically acceptable salt thereof, are provided as separate compositions for administration at the same time or at different times.

30. The method according to claim 29, wherein Aplidine, or a pharmaceutically acceptable salt thereof, and Gemcitabine, or a pharmaceutically acceptable salt thereof, are provided as separate compositions for administration at different times.

31. A kit for administering Aplidine, or a pharmaceutically acceptable salt thereof, in combination with Gemcitabine, or a pharmaceutically acceptable salt thereof, comprising a dosage form of Aplidine, or a pharmaceutically acceptable salt thereof, and/or a dosage form of Gemcitabine, or a pharmaceutically acceptable salt thereof, and printed instructions for administering both drugs in combination.
